(19) United States
(12) Patent Application Publication
(10) Pub. No.: US 2013/0267803 A1
(43) Pub. Date: Oct. 10, 2013

(54) PULSE OXIMETER WITH INTERNAL STERILIZATION AND METHODS

(71) Applicant: KABLOOE DESIGN, INC., Minneapolis, MN (US)

(72) Inventor: Thomas E. Kramer, Minneapolis, MN (US)

(73) Assignee: Kablooe Design, Inc., Minneapolis, MN (US)

(21) Appl. No.: 13/859,685

(22) Filed: Apr. 9, 2013

Related U.S. Application Data
(60) Provisional application No. 61/621,646, filed on Apr. 9, 2012.

(51) Int. Cl. A61B 5/1455

(52) U.S. Cl. CPC A61B 5/14551
USPC 600/323

(57) ABSTRACT

A pulse oximeter is described herein. Specifically, The invention provides a non-invasive optical sensor comprising a hinged housing having a pair of opposed faces and an internal self-sterilizing source.

### FIELD

The invention relates to pulse oximeters and method of using pulse oximeters.

### BACKGROUND

Pulse oximeters are widely used across clinical settings, such as operating rooms, emergency rooms, post anesthesia care units, critical care units, outpatient surgery and physiological labs, to name a few. Use in these settings expose pulse oximeters to the potential risks of contamination and the resulting spread of nosocomial (hospital-acquired) infections. While a low-level disinfection protocol of alcohol wipes, dilute bleach scrubs or distilled water wipes can be effective when done correctly, pulse oximeters may still be at risk for bacteria including MRSA (methicillin resistant staphylococcus aureus). MRSA causes skin infections and can occasionally spread to almost any other organ in the body, sometimes with life-threatening potential. It is therefore a priority among medical care facilities to prevent contamination of medical equipment by foreign and infectious materials and to prevent the spread of nosocomial infections. A self-sterilizing pulse oximeter advantageously provides a sterile environment without interfering with functionality and capability.

This invention relates generally to sensors for sensing physiological functions in a human being and, more particularly, to an oximetry device for positioning and holding an optical sensor adjacent a finger or other body extremity. Various non-invasive techniques have been developed for sensing physiological functions in a human medical patient. Such non-invasive techniques have the advantage of avoiding physical penetration of the skin. This substantially reduces the risks of infection, trauma and minimizes patient discomfort.

One well known technique for non-invasively sensing physiological functions involves passing infrared or visible light through a portion of a patient's body. By measuring the relative absorption at various wavelengths, information regarding the patient's physiological functions can be derived. Such “optical sensing” is particularly useful in pulse oximetry wherein the instantaneous relative oxygenation of a patient's arterial blood is determined by passing light through a blood-perfused portion of the patient's body (e.g., the finger) and instantaneously measuring the relative absorption at one or more selected wavelengths. Typically, one or more optical sources (e.g., light emitting diodes or “LED's”) are positioned on one side of the finger, and one or more optical detectors (e.g., photodiodes) are located on the opposite side. A clip device holds both the sources and detectors in their respective, proper positions.

Because it is sometimes necessary to monitor a physiological function for hours, days or even weeks at a time, much consideration must be given to the means by which optical sensing devices are attached or coupled to the patient. On the one hand, a firm means of attachment is desirable in order to ensure continual and reliable monitoring. On the other hand, a too firm means of attachment can cause considerable discomfort, particularly if long term monitoring is involved. Consideration, therefore, must be given to reliability and performance consistent with patient comfort.

Still further consideration should be given to the avoidance of infection and disease transfer. Although non-invasive monitors do not, as a rule, physically enter a patient's body or bloodstream, cleanliness is nevertheless recognized as essential in preventing the spread of disease. Although single-use, disposable devices are one well known way of ensuring sterility and avoiding disease transfer, the disposal of medical waste material is a growing problem, and the costs and waste associated with discarding complex, sophisticated devices after only a single use are becoming increasingly difficult to justify. Preferred devices are those that can be economically manufactured and easily cleaned for multiple use.

One known clip for mounting an optical sensor on a patient's finger is shown in U.S. Pat. No. 4,685,464. In such a clip, a pair of deformable pads, on which are mounted, respectively, a light source and a light detector, in turn are mounted on and adhered to the opposed faces of a rigid, hinged, clothespin-like housing. Although effective, the permanently affixed pads make effective cleaning of the surface somewhat difficult and inefficient.

Another known optical sensor is shown in U.S. Pat. No. 5,438,986. In such an optical sensor the components can be readily disassembled for cleaning and repair. Although, this permits a more convenient process to clean various portions of an optical sensor, the process of disassembling an optical sensor to sanitize the components before subsequent use is inefficient.

Another known sanitation method is shown in U.S. Pub. No. 2002/0146343. In such a method, sterilization is performed by irradiating a surface with ultra-violet light from an external source. The sanitation device can be portable or a fixed device. Although this device is described to be available to sanitize a number of medical devices, each device must be capable of being exposed from the UV light from an external source.

### BRIEF DESCRIPTION OF THE DRAWINGS

The invention will be further explained with reference to the appended Figures, wherein like structure is referred to by like numerals through the several views, and wherein:

FIG. 1 illustrates a schematic right side elevation view in an open position.

FIG. 2A illustrates a schematic of a left side view of a pulse oximeter device.

FIG. 2B illustrates a schematic of a right side view of a pulse oximeter device.

FIG. 3A illustrates a schematic right side perspective in an closed position.

FIG. 3B illustrates a schematic left side elevation view in an open position.

### DETAILED DESCRIPTION

The invention provides novel products and methods. Specifically, the invention provides a non-invasive optical sensor comprising a moveable housing having a pair of opposed faces and a self-sterilizing source.

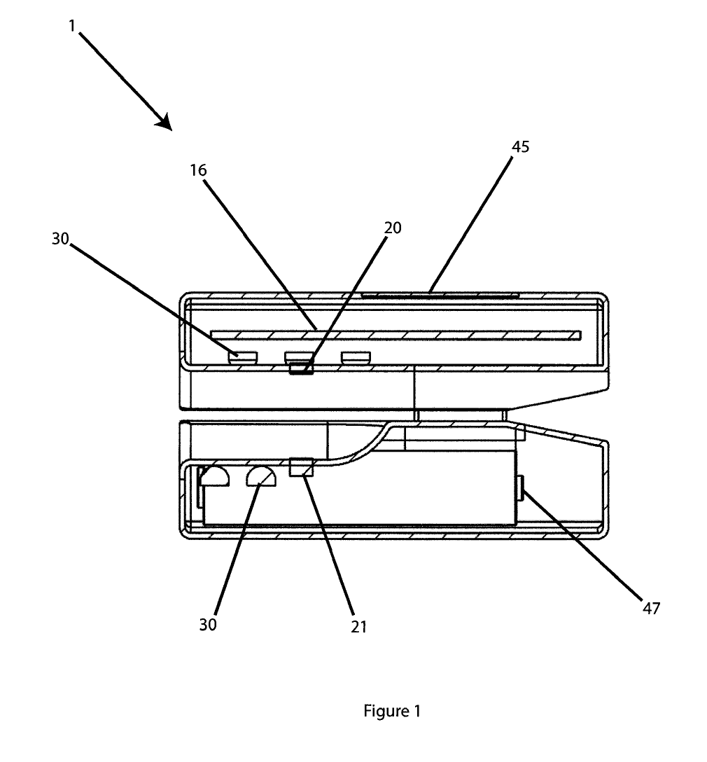

Referring now to FIG. 1, a self-sterilizing pulse oximeter 1 of the invention generally comprises a housing 10, a set of sensors 20 and 21 and a sterilizing light source 30. Sensors 20 and 21 and sterilizing light source 30 are disposed within the interior portion of the housing 10.

Housing 10 of oximeter 1 is formed from a versatile material that is medically safe, does not degrade upon exposure to ultra-violet irradiation and provides structural reliability. In one example embodiment, housing 10 is made with silicone or polyurethane. Housing 10 may also be formed of a rigid, durable, injection molded plastic such as acrylonitrile butadiene styrene (ABS) or ABS/polycarbonate. In another embodiment, housing 10 is formed of a rigid, durable, non-deformable plastic such as rigid PVC, and is preferably of solid construction, to avoid slippage and improve patient feel.

In other example embodiments, housing 10 also comprise synthetic materials such as various types of rubbers, neoprene, nylon, PVC, polystyrene, polyethylene, polypropylene and biocompatible polymers.

Housing 10 of oximeter 1 generally comprises a first portion 12 and second portion 14. First portion 12 and second portion 14 are moveable with respect to one another. First portion 12 and second portion 14 also provide a means to sufficiently receive a patient extremity.

First portion 12 and second portion 14 are generally hollow to provide a cavity to dispose therein sensors 20 and 21 and a sterilizing light source 30. In an alternative embodiment, at least one of first portion 12 and second portion 14 is hollow. In such an embodiment, the sensor or sensors and a sterilizing light source are integrally related within the housing surface.

In an alternative embodiment, housing 10 comprises a single portion or member (not shown) that is flexibly moveable so as to provide a clamping means around a patient extremity. This single portion embodiment provides for two opposing surfaces, such that the patient's extremity can be placed between a first patient contact portion and second patient contact portion.

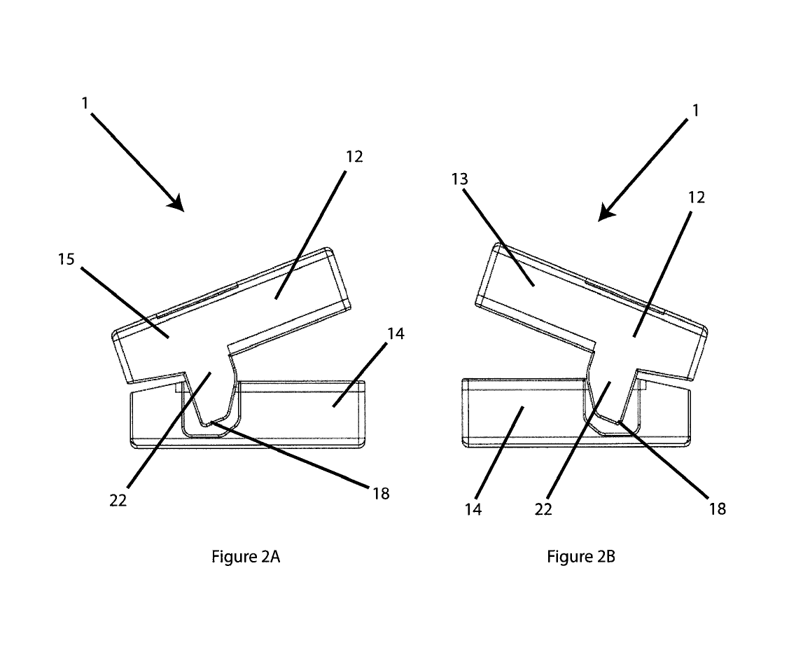

Referring now to FIGS. 2A and 2B, first portion 12 and second portion 14 are joined by a hinge mechanism 18. In an example embodiment, first portion 12 includes an downwardly projecting tab 22 having an inwardly projecting pin (not shown). The inwardly projecting pin is positioned to couple tab portion 22 to the second portion 14. The second portion 14 includes a slot (not shown) for receiving pin. Hinge mechanism 18 is located on a first side 13 (FIG. 2B) and a second side 15 (FIG. 2A) of housing 10.

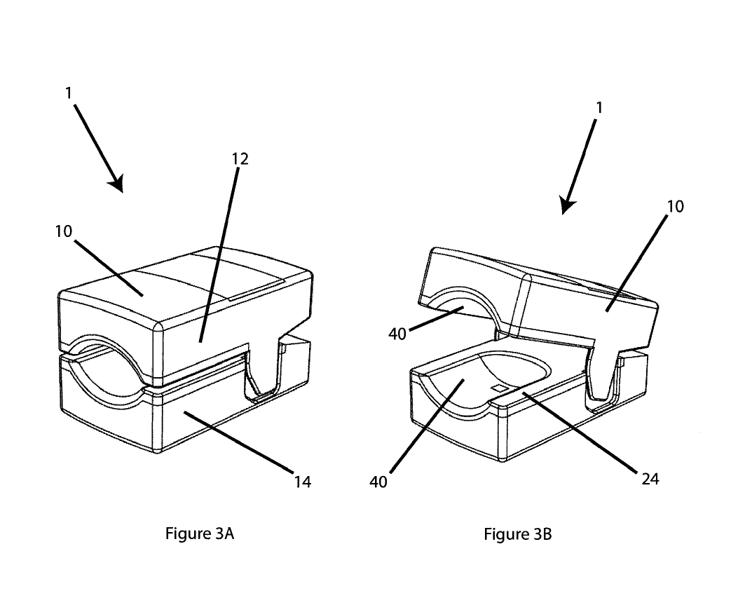

Referring now to FIGS. 1, 3A and 3B, oximeter 1 provides a means to position housing 10 on a person in need thereof. A patient contact surface 40 conforms generally to the shape of the patient extremity to provide for temporary fastening. Patient contact surface 40 can be curved, as shown in FIG. 3A and 3B.

In alternative embodiments, the patient contact surface is parallel to the opposed patient contact surface. Patient contact surface 40 is shaped to provide a substantially similar surface to the patient surface being contacted. For example, a horizontal configuration (not shown) may be used to attach to a patient extremity such as an earlobe. In an alternative embodiment, patient contact surface 40 may be curved to provide a contact portion for a patient extremity such as a finger or toe. The patient contact surface geometry is determined to provide adequate securement to a patient, while also providing a sufficient amount of comfort to the patient when attached. The particular geometry of the patient contact surface is well known by one of ordinary skill in the art.

Referring still to FIGS. 3A and 3B, patient contact surfaces 40A and 40B are made of a transparent material capable of allowing a sufficient amount of sterilizing light to pass through. Although transparent plastics are preferred, any substance capable of allowing sterilizing light source 30 to deliver light sufficient to sterilize patient contact surfaces 40 is within the scope of the invention.

In alternative embodiments, patient contact surface 40 may have a friction enhancing material applied to a portion of patient contact surface 40 so long as the friction enhancing material does not block the internal sterilizing light source 30. For example, a thin layer of silicone rubber can be included on the patient contact surface 40.

Optical sensors 20 and 21 comprise at least one optical source and at least one optical detector. The optical source and the optical detector are each preferably coupled to patient contact surface 40. The optical source of the invention is placed opposite the optical detector to thus enable highly accurate measurement of a patient's physiology, including blood-oxygen concentration.

In one embodiment, optical source 20 is attached to first portion 12 and the optical detector 21 is attached to second portion 14. However, the optical source may be attached to second portion 14 and the optical detector may be connected to first portion 12.

Referring to FIG. 1, housing 10 also has an internal circuit board 16 disposed within the internal portion or cavity of the housing 10. Although the embodiment describes internal circuit board 16 being disposed along the upper interior of first portion 12 of housing 10, the location within housing 10 can be placed in any number of locations. Optimal placement of an internal circuit board within a housing would be apparent to one of ordinary skill in the art.

A wire (not shown) is used to electrically connect self-sterilizing pulse oximeter 1 to sensors 20 and 21 of the invention. The wire provides for communication between sensors 20 and 21, internal circuit board 16, and display 45. Although display 45 is disposed along the upper exterior portion of first portion 12, display 45 may be placed in any position allowing the user to see display 45. However, display 45 may also be separate from housing 10 and be coupled through a wired means. Alternatively, display 45 may also be in wireless communication with sensors 20 and 21 and internal circuit board 16.

The sterilizing light source 30 is placed along the back portion of the patient contact surface 40. In one embodiment, as shown in FIG. 1, the sterilizing light source 30 is an ultraviolet light-emitting diode (LED). The ultraviolet LED is positioned along the patient contact surface to provide ultraviolet sterilization to the opposing patient contact surface.

It should be appreciated that the precise number and placement of the ultraviolet LED(s) used for sterilization may depend on the specific application of the self-sterilizing pulse oximeter. Furthermore, an embodiment described herein includes an ultraviolet LED light as sterilizing light source 30, the sterilizing light source 30 may include UV LEDs, UV fluorescent bulbs, or any combination of UV light source. Additionally, sterilizing light source 30 may include other forms of energy such as red light, near infrared light and microwave.

Housing 10 further includes a spring (not shown), which is coupled to first portion 12 and second portion 14. The spring of the housing 10 generally provides for pivoting movement between first portion 12 and second portion 14. Ordinarily, as described in FIG. 3A, the bias of the spring pulls first portion 12 and second portion 14 thereby minimizing the distance between first portion 12 and second portion 14.

Now referring to FIGS. 1 and 3B, self-sterilizing pulse oximeter 1, further comprises a switch 24. Switch 24 provides a means of activating sterilizing light source 30, when the self-sterilizing pulse oximeter 1 is in a closed position, as shown in FIG. 1. The switch activates an internal monitor that will provide a sufficient amount of sterilizing light to patient contact surface 40. Switch 24 further provides a safety feature to prevent the irradiation of a patient extremity contained within as switch 24 deactivates sterilizing light source 30 when self-sterilizing pulse oximeter 1 is in a open position as shown in FIG. 3B. Switch 24 further prevents inadvertent irradiation of surrounding persons when self-sterilizing pulse oximeter 1 is in an open position. When switch 24 is turned off, self-sterilizing pulse oximeter 1 can be used to monitor a patient physiology.

The source of power for self-sterilizing pulse oximeter can be any source sufficient to provide power to operate sensors 20 and 21, internal circuit board 16 and display 45. In one embodiment, the power source is a battery 47. Battery 47 provides the convenience of providing a portable device.

Batteries, such as disposable batteries, offer a cost advantage, are typically long-lasting and are easily replaceable. Alternatively, rechargeable batteries are also within the scope of the invention. Rechargeable batteries although a bit more expensive up front, they in turn provide the ability to recharge the batteries without having to dispose of them and thus they are more environmentally friendly than disposable batteries as they create less waste. In a related embodiment, solar cells on the outside of housing 10 are used. In an alternative embodiment, the invention uses a plug to access power from an electrical outlet. Therefore, depending on the manner of using the oximeter and to what extent and the frequency you will be using it will affect your decision as to which power source or combination of power sources to use.

When the invention is in use, self-sterilizing pulse oximeter 1 provides a means of using ultraviolet light to sterilize patient contact surface 40. The sterilization wavelength of approximately 250 nm is utilized. Although wavelengths of about 250 nm is suitable for reducing the bacterial concentration on patient contact surface 40, any wavelength suitable for eliminating or diminishing bacteria on a surface of the pulse oximeter 1 may be utilized.

One consideration to be made when self-sterilizing pulse oximeter 1 is in use is the safety in using a sterilizing light source, such as UV light. UV radiation can be hazardous to people if there is prolonged exposure to skin, leading to sunburn and other skin conditions, as well as potential injury to the eyes if exposed to the radiation. For this reason, the self-sterilizing pulse oximeter may include safety features that selectively activate and deactivate sterilizing light source 30. These features may include a timed irradiation or a lock to prevent opening self-sterilizing pulse oximeter 1 when being sterilized. Self-sterilizing pulse oximeter 1 may also be configured to modify or modulate the intensity or wavelength of the sterilizing light source according to any number and type of sterilization criteria. As previously mentioned safety features such as providing the deactivation of sterilizing light source 30 housing 10 is in an opened position, as described in FIG. 3B.

While the invention has been illustrated and described with particularity in terms of preferred embodiments, it should be understood that no limitation of the scope of the invention is intended thereby. The scope of the invention is defined only by the claims hereby appended. It should be understood that variations of the particular embodiments described herein incorporating the principles of the invention will occur to those of ordinary skill in the art and yet will be within the scope of the appended claims.

What is claimed is:

1. A pulse oximeter comprising:
a housing having a first portion and a second portion, wherein said first portion and said second portion are movable with respect to one another sufficient to receive a patient extremity therebetween, said housing having at least one patient contact surface for which to dispose said patient extremity thereon;
at least one sensor means disposed in said housing to sense patient physiology through said patient extremity; and
a sterilizing light source disposed within said housing for sterilizing said patient contact surface.

2. The pulse oximeter of claim 1, having means therein for communicating with a display system on said housing for monitoring said patient physiology.

3. The pulse oximeter of claim 1, wherein said sterilizing light source is selected from a group consisting of ultraviolet light-emitting diodes (LED), ultraviolet florescent lamps and ultraviolet bulbs.

4. The pulse oximeter in claim 1, wherein said at least one sensor means having an optical source and an optical detector.

5. The pulse oximeter in claim 4, further comprising a circuit board, electrically coupled with said optical source and said optical detector, wherein said circuit board transmits signals to a display.

6. The pulse oximeter in claim 5, wherein said circuit board transmits a wireless signal.

7. The pulse oximeter in claim 5, wherein said circuit board transmits a wired signal.

8. The pulse oximeter in claim 1, said patient contact surface of said first portion and said second portion are shaped to substantially conform to an extremity of the patient's body.

9. The pulse oximeter in claim 1, wherein said first portion and said second portion are configured to be at least partially transparent.

10. The pulse oximeter in claim 1, wherein said optical source is a light-emitting diode said light-emitting diode adapted to transmit red and near-infra red light.

11. The pulse oximeter in claim 1, further comprising a power source.

12. The pulse oximeter in claim 11, wherein said power source is selected from the group consisting of a DC source, an AC source and a solar power source.

13. The pulse oximeter in claim 11, wherein said power source is selected from the group consisting of a battery, an electrical outlet and a solar cell.

14. A pulse oximeter comprising:
a housing comprising a first portion and a second portion, wherein said first portion and said second portion being moveable and each of said first portion and second portion having a patient contact surface;
at least one sterilizing source, wherein said sterilizing source being interiorly disposed within said housing,
at least one optical source, and
at least one optical detector, wherein said optical source being disposed on a first patient contact surface of said housing facing toward said optical detector and said optical detector being disposed on a second patient contact surface of said housing facing toward said optical source.

15. A pulse oximeter comprising:
a housing comprising a first portion and a second portion, wherein said first portion and said second portion being moveable and each of said first portion and second portion having a patient contact surface;
at least one sterilizing source, wherein said sterilizing source being interiorly disposed within said housing,
at least one optical source, and
at least one optical detector, wherein said optical source being disposed on a first patient contact surface of said housing facing toward said optical detector and said optical detector being disposed on a second patient contact surface of said housing facing toward said optical source, and
a means for sterilizing a portion of a pulse oximeter with an interiorly disposed sterilizing source.

16. A method of using a self-sterilizing pulse oximeter comprising:
A. Positioning a housing of the self-sterilizing pulse oximeter in a closed position,
B. activating a sterilizing light source, wherein said sterilizing light source is disposed within said housing, and
C. irradiating a patient contact surface with said sterilizing light source.
